(19) United States
(12) Patent Application Publication
(10) Pub. No.: US 2024/0181695 A1
(43) Pub. Date: Jun. 6, 2024

(54) MOLDING WITH SELF RISING MATERIALS

(71) Applicant: Piana Nonwovens, LLC., Cartersville, GA (US)

(72) Inventors: Michael DeFranks, Cartersville, GA (US); Andy Hollis, Cartersville, GA (US); Eric McCann, Cartersville, GA (US)

(21) Appl. No.: 18/527,655

(22) Filed: Dec. 4, 2023

Related U.S. Application Data
(60) Provisional application No. 63/429,723, filed on Dec. 2, 2022.

(51) Int. Cl. B29C 61/04; B29C 33/04; B29K 105/00; B29K 105/12

(52) U.S. Cl. CPC B29C 61/04; B29C 33/048; B29K 2105/128; B29K 2105/256

(57) ABSTRACT

Three dimensional molded objects are produced from a plurality of expandible elements deposited in a mold by heating and moving the expandible elements during heating in the mold. The expandible elements have at least one layer of vertically lapped material held in a pre-compressed state during or before they are deposited in the mold. Movement is achieved by physically moving the mold continuously or periodically during heating, by blowing air or other gas into the mold continuously or periodically during heating, or by heating and depositing and moving the expandible objects at the same time.

### CROSS-REFERENCE TO RELATED APPLICATIONS

This application claims priority to U.S. Provisional Application 63/429,723 filed on Dec. 2, 2022. The complete contents thereof is herein incorporated by reference.

### FIELD OF THE INVENTION

The disclosure generally pertains to molding of components for vehicles such as cars, boats, planes, etc. or furniture such as office seats, sofas, bedding, etc. which employ vertically lapped (perpendicular-laid) nonwoven materials (VLAP materials).

### BACKGROUND

U.S. Pat. No. 3,607,500 to Feld describes molding an article from a compressed fibrous web with a thermoplastic binder. Feld explains that the compressed fibrous webs can be made from a fibrous web material sprayed with thermoplastic binder which is inserted into a press and compressed with application of heat. Then, thermoplastic in the web is permitted to solidify to form a wafer thin material with compressed fibers. This wafer thin material can be shipped to a destination where molded articles are desired, and it can be inserted into a mold and heated. In the mold, the fibers expand, perhaps to its original thickness in some parts, and the mold is filled with the fibrous material. On cooling in the mold, a molded article is formed from the fibrous material and thermoplastic binder.

European Application 0538047 to Earl describes a nonwoven material made from high loft fibers and lower temperature melting synthetic fibers. Earl describes a process similar to Feld where an interim compressed product can be made by heating nonwoven to melt the synthetic fibers and compressing the material, followed by allowing the material to cool with the synthetic fibers re-solidifying. This interim product may then be more easily transported, and then used to form products in a mold.

Neither Feld nor Earl contemplate combining the fibrous material with other materials, or making self-rising boards from several layers of compressed fibrous webs which are stacked and/or are laminated together.

Self-rising boards made from VLAP materials are described in detail in U.S. Pat. No. 11,472,132 to Piana, and the complete contents thereof is herein incorporated by reference. In summary, the self-rising boards, which may be referred to as blanks, are formed by compressing one or more nonwoven materials, preferably including nonwoven VLAP materials under pressure after melting binder material (a polymer) used to hold fibers of the nonwoven materials together. Application of heat during the compressing process can be done using hot air, ovens, induction coils, infrared or other means for applying heat energy. While in its compressed state, the nonwoven is cooled in order for the binder material to re-harden (solidify). This may be accomplished simply by removing the heat and/or by blowing cool air through the blanks. The blanks thus formed will typically have a height dimension substantially less than the nonwoven starting material (e.g., 10% to 50% as thick as the pre-compressed assembly; however, differing thicknesses may be used, with the chief requirement being that the compressed dimension is at least smaller than the original dimension of the nonwoven (be it height, width, or length), and preferably 50% smaller, 60% smaller, 70% smaller, 80% smaller, 90% smaller, etc.). In the compressed state, the fibers of the nonwoven cross over or next to each other at different locations than in the pre-compressed state, and the binder material, once re-solidified, holds the fibers together at these different locations. If desired, the blanks can be laminated together to form boards. Or the blanks can be laminated together with non-expandable materials such as foams, fabric (e.g., knitted material), rubber, metal, metal alloy, polymeric, ceramic, and paper materials. If desired, these boards may be cut to form parts which have specific sizes and shapes with a CNC machine, milling machine, scissors, or other suitable device. The blanks, boards, or parts are all “expandable substrates” in that they can expand upon heating to fill or partially fill a mold to make a three dimensional object of interest.

The multilayer self-rising boards of U.S. Pat. No. 11,472,132, can include a plurality of layers of VLAP materials laminated together where each layer is made of the same or different fibers and/or binder materials such that the layers have the same or different self rising properties. For example, one or more layers could expand more than the other layers depending upon the configuration of the VLAP (e.g., how high the fibers extend) or its constitution (e.g., the fibers themselves or the binder material). In addition, the multilayer self-rising boards of U.S. Pat. No. 11,472,132, can include at least one layer of VLAP material together with a plurality of layers which include non VLAP materials (e.g., cross lapped non-wovens) or even non-expandible layers (e.g., paper, metal, polymer, ceramic, foam, etc.). The multilayer self rising boards offer significant advantages to the Feld wafers and the Earl interim pieces, in that the self rising boards can have different materials, and hence different properties being provided, in different layers.

A need exists for easily molding complex structures (e.g., chairs with defined regions for different parts of a seat and different parts of a back).

### SUMMARY

According to the invention, three dimensional objects of complex shape (e.g., having different thicknesses at different locations, contours, etc.), are formed from self rising materials (those having at least a portion made of a VLAP material in a compressed state which can be expanded towards a pre-compressed, enlarged state on melting a binder material during molding) by using a plurality of expandible elements within the mold, and moving the expandible elements throughout the interior of the mold during the heating procedure. This is accomplished by depositing within the interior volume of the mold the a plurality of expandible elements, where the expandible elements are comprised of at least one layer of compressed vertically lapped nonwoven material having binder polymer holding fibers of the vertically lapped nonwoven material in a compressed state, and wherein each of the plurality of expandible elements is smaller than at least one tenth of the interior volume of the mold, and more preferably less than one fiftieth, or one hundredth, or one one thousandth the mold volume. Good results may be obtained with expandible elements that are 10 mm, or 5 mm, or 2 mm or smaller on a side. While in the mold, the plurality of expandible elements are heated so as to melt the binder polymer and allow the fibers of the vertically lapped nonwoven material to expand to an expanded state. During heating or at least at periodic times during heating, the plurality of expandible elements are moved to different locations in the interior volume of the mold. This can be accomplished by blowing air into the mold, by physically moving the mold itself around, or by a process where the elements are heated while they are being deposited within the mold. Cooling the plurality of expandible elements after heating and moving is performed so that the binder polymer solidifies and holds the fibers in the expanded state, and holds adjacent expandible elements of the plurality of expandible elements together so as to form a molded element. Then, the molded element is retrieved from the interior volume of the mold (e.g., by opening the mold). During fabrication of the three dimensional molded element, the amount of the plurality of expandible elements deposited into the mold should be sufficient to fill the interior volume of the mold when the molded element is formed during the cooling step.

### DESCRIPTION OF THE DRAWINGS

FIG. 1 is a schematic showing molding of a self rising board in a mold having a complex (i.e., non-uniform) interior:

FIG. 2 is a schematic showing a self rising board as shown in FIG. 1 pelletized to make a plurality of expandible elements;

FIG. 3 is a schematic showing filling the mold of FIG. 1 with the plurality of expandible elements of FIG. 2 to make a three dimensional object of relatively uniform density;

FIG. 4 is a schematic showing one example for agitation or movement of the plurality of expandible elements during molding;

FIG. 5 is a schematic showing another example for agitation or movement of the plurality of expandible elements during molding; and

FIG. 6 is a schematic showing yet another example for agitation or movement of the plurality of expandible elements during molding.

### DESCRIPTION

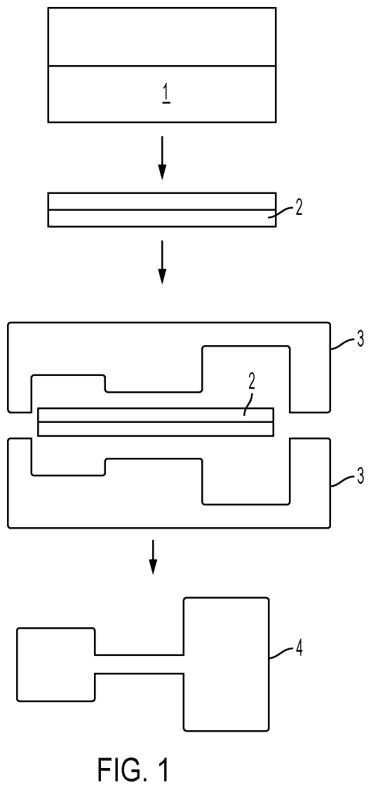

When molding complex shapes from self rising boards (e.g., multilayer materials where at least one of the layers is a compressed nonwoven), challenges are raised with respect to having a controlled density throughout the shaped article produced. For example, as illustrated in FIG. 1, a self rising board 2 is insertable into a mold 3, such as a steam mold), and is heated to make a three dimensional object 4 by having the self rising board 2 expand during heating to fill the interior volume of the mold 3. By heating the self rising board 2, the binder material is melted, and the pre-compressed fibers are released from the compressed state and are allowed to move to an uncompressed or expanded state. Then, by cooling, the binder material solidifies to hold fibers together while they are in their expanded state. In the example shown in FIG. 1, the self rising board 2 when inserted in a mold 3 having a complex shape (multiple and different thicknesses, as well as contours, in the interior mold volume), may have difficulty in forming the three dimensional objects 4 of relatively uniform density because the areas or regions in the mold where the fibers in the self rising board are permitted to expand to a greater degree (e.g., the ends of the mold shown in FIG. 1) will tend to be less dense, and the areas or regions in which the fibers are restricted from significant expansion (e.g., the central section in the mold shown in FIG. 1) will tend to be more dense. Thus, the three dimensional molded object 4 may have a variable density from region to region.

The self rising board 2 may comprise one or more layers of compressed VLAP materials made by compressing a multilayered material 1, at least one of which is a layer of VLAP material. The self rising board 2 is made by heating the multilayered material to a temperature sufficient to melt binder material (e.g., fibers of a thermoplastic polymer such as polyester, etc.), followed by cooling to a point to solidify the binder material so that it joins together fibers of the VLAP material at locations that are compressed relative to their initial locations (i.e., the relative locations of the fibers in a pre-compressed state). In the practice of this invention, and that described relative to FIG. 1, all or portions of the nonwoven layers disclosed herein are vertically lapped. A “nonwoven” is a manufactured sheet, web, or batt of natural and/or man-made fibers or filaments that are bonded to each other by any of several means. Manufacturing of nonwoven products is well described in “Nonwoven Textile Fabrics” in Kirk-Othmer Encyclopedia of Chemical Technology, 3rd Ed., Vol. 16, July 1984, John Wiley & Sons, p. 72.about.124 and in “Nonwoven Textiles”, November 1988, Carolina Academic Press.

Nonwovens are commercially available from a number of manufacturers. As used herein, the term “vertically lapped” is meant that one or a plurality of materials is in the form of a web that has been folded in on itself in a corrugated fashion to produce a three-dimensional structure that has been thermally bonded and often is referred to as perpendicular laid. A “vertical lapper” is also referred to as a “STRUTO” or a “V-LAP” and some examples of machinery which may be used to make vertically lapped nonwovens for use in the invention are herein incorporated by reference (WO 2015176099 to Cooper and U.S. Pat. No. 7,591,049 to Cooper). Vertically lapped nonwovens are higher in compressional thermal resistance and lighter in weight than those made of fibers horizontally lapped, horizontally cross-lapped, horizontally woven and/or polyurethane foams. The vertically lapped nonwoven process takes a carded fiber web and laps it vertically (i.e. pleating) rather than horizontally laying the fibers. The size, shape and arrangement of the material of nonwovens may vary widely as long as nonwovens are made directly from separate fibers, molten plastic or plastic films, but not made by weaving or knitting. In an exemplary embodiment, the nonwoven is manufactured by hot-air thermal bonding using low-melt and/or elastomeric binder fibers. The binding fibers serve to mix readily with the other fibers of a nonwoven, e.g. staple fibers, and to melt on application of heat and then to re-solidify on cooling to hold the other fibers in the nonwoven together. In some applications, the binding fibers might have a core-sheath configuration where the sheath melts on application of heat and functions to hold the other fibers of the nonwoven together.

In particular, the nonwoven can have a basis weight ranging from 0.1-5.0 oz/ft²; however, the basis weight of the nonwoven can vary widely depending on the intended application and desired characteristics of the nonwoven. A plurality of fibers, from natural to synthetic, may be used for manufacture of vertically lapped nonwovens. The nonwoven can include combinations of two or more different natural fibers; two or more different man-made synthetic fibers; blends containing one or more natural fibers and one or more man-made fibers. Exemplary fibers which can be used in the practice of the invention include but are not limited to: cotton, kapok, flax, ramie, kenaf, abaca, coir, hemp, jute, sisal, rayon, bamboo fiber, Tencel®, and Modal® fibers, glass fibers, basalt fibers, Kevlar® fibers, aramid fibers, polyester fibers (e.g., which can function both as a binder fiber but, depending on the polyester, as part of the nonwoven blend), wool (which may be obtained, for example, from one of the forty or more different breeds of sheep, and which currently exists in about two hundred types of varying grades), silk, rayon (a man-made fiber that may include viscose rayon and cuprammonium rayon), acetate (a man-made fiber), nylon (a man-made fiber), acrylic (a man-made fiber), polyester (a man-made fiber), triacetate (a man-made fiber), spandex (an elastomeric man-made fiber such as Lycra®), polyolefin/polypropylene (man-made olefin fibers), microfibers and microdeniers, lyocell (a man-made fiber), vegetable fiber (a textile fiber of vegetable origin, such as cotton, kapok, jute, ramie, polylactic acid (PLA) or flax), vinyl fiber (a manufactured fiber), alpaca, angora, carbon fiber (suitable for textile use); (t) glass fiber (suitable for textile use), raffia, ramie, vinyon fiber (a manufactured fiber), Vectran® fibers (manufactured fiber spun from Celanese Vectra® liquid crystal polymer), and waste fiber. Fibers are commercially available from sources known by those of skill in the art, for example, E.I. Du Pont de Nemours & Company, Inc. (Wilmington, Del.), American Viscose Company (Markus Hook, Pa.), Teijin Frontier Co., Ltd. (Osaka, Japan), Tintoria Piana USA (Cartersville, Ga.), and Celanese Corporation (Charlotte, N.C.).

Exemplary types of polyesters which may be used in the practice of the invention include, but are not limited to PET (polyethylene terephthalate), PTT (polytrimethylene terephthalate), and PBT (polybuthylene terephthalate).

Nonwovens useful in the practice of the invention can also be formed using composite fibers, sometimes referred to as sheath-core fibers. Binder fibers used to produce nonwovens useful in the practice of this invention include sheath-core fibers, where the sheath is polyester or some other low melting temperature material.

As described above, the vertically lapped structural layer may comprise a combination of staple and binder fibers, such as a polyester staple fiber (optionally, a hollow conjugate fiber) and a polyester binder fiber. The binder fibers have a melting temperature that is below the melting or decomposition temperature of the one or more other fibers, e.g., binder fibers typically have a melting temperature of 80-200° C. (polyesters are typical examples of binder fibers used in the production of nonwovens (examples of elastic polyester binder fibers include ELK®, E-PLEX®, and EMF type high elastic LMF are commercially available from Teijin Limited, Toray Chemical Korea Inc., and Huvis Corporation, respectively)). Once the binder fibers are melted, they will generally track along the outsides of the one or more other fibers, and, on cooling, will harden to produce the nonwoven which is essentially a mass of the one or more other fibers with adjacent fibers held together at various locations throughout the nonwoven by binder material which results from melting and re-hardening of the binder fibers. These nonwovens are often referred to as thermobonded nonwovens. The vertically lapped structural layer may have 60-80% by weight binder material, with 20-40% by weight staple fibers. In some embodiments, the length of the staple fibers may be from 45-75 mm, e.g. 50-55 mm. The weight of the fabric may be between 500-1500 GSM. The loft may be from 25-90 mm.

The nonwoven layers described herein can be formed using fibers that are treated with chemicals (e.g., dyes (for coloring of some or all of the fibers), fire retardant chemicals (e.g., phosphates, sulfates, silicates, etc.), scent's (perfumes, etc.), topical additives such as phase change material particles, talc, carbon nanotubes, etc.). Alternatively, a plurality of chemicals (e.g., dyes, scents, fire retardant chemicals, addition of microparticles, etc.) may be used to treat the nonwoven after completion of the final assembly of a structure.

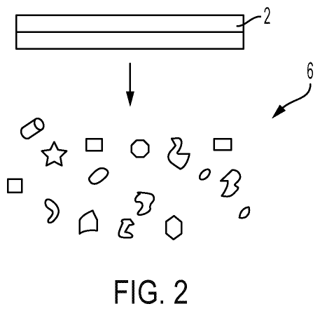

To address the variable density problem, the self rising board of FIG. 1 may be pelletized to form VLAP beads or pieces 6, hereinafter referred to as a plurality of expandible elements 6, as shown in FIG. 2. This can be accomplished by a number of means including cutting, hole punching, or otherwise separating a self rising board as shown in FIG. 1 into a plurality of self rising beads, pieces or elements. These self rising beads, pieces or elements 6 can be of any shape (cylindrical, square, polygonal, nonuniform, etc.) and, the shapes of the beads, pieces or elements do not need to be the same. Furthermore, the self rising beads, pieces or elements can have a maximum dimension (e.g., largest diameter, largest length of side, length of diagonal, etc.) that is between ⅛ inch to 2 inches (in some embodiments it can be ¼ inch to one inch) (1-10 mm, and 1-5 mm in some applications), and, as with the shape, the maximum dimension can be different for different beads, pieces, or elements in the group which is created. These beads, pieces or elements can be created with a hole punch or with scissors, shears, or by other means.

Furthermore, depending on the application, the self rising board from which the beads, pieces or elements are made can be constructed with some of the layers being non VLAP material (e.g., they could, for example, include carbon nanotubes, pieces of leather, woven glass fibers (as well aramid and other fibers). Thus, each of the beads, pieces, or elements would include the non VLAP material together with layers of VLAP material.

While the expandible elements 6 are described as being pelletized from the expandible board 2 by a pelletization process, it should be understood that these expandible elements 6 can also be made individually. That is, each expandible element can have a multilayer structure with at least one layer of VLAP material in a compressed state, as described above.

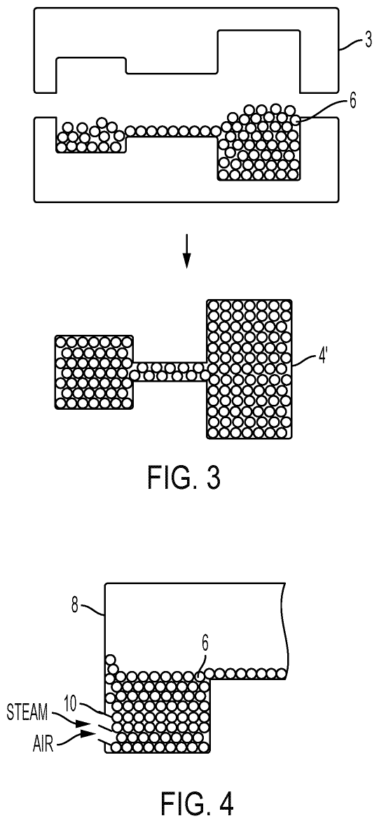

With reference to FIG. 3 the mold 3 of FIG. 1 can be partially filled with the plurality of expandible elements 6, and, upon heating the plurality of expandible elements 6 the elements would expand to fill the mold 3 and then, upon cooling, three dimensional object 4′ produced would generally have a more uniform density than that which can be produced with the larger boards 2 as shown in FIG. 1. The areas of the mold 3 which are larger (the ends) than the areas which are thinner (the middle), will contain relatively more of the beads, pieces, or elements. Thus, at least in some applications, using the plurality of expandible element 6 lead to a three dimensional part of more uniform density after the self rising beads, pieces or elements are permitted to have their fibrous parts expand under the application of heat while inside the mold. In addition, in some embodiments, it will be beneficial to pre-heat the VLAP beads, pieces or elements 6 prior to placing them in the mold 3. For example, they could be heated to a point up to or just below the melting temperature of the thermoplastic polymer. Such a pretreatment may enable the VLAP beads, pieces or elements to be more pliable and to begin expansion in size, and in some cases may make them easier to distribute in the mold. Furthermore, the pre-heat treatment can also make the VLAP beads, pieces or elements more uniform in terms of temperature before steam treatment with the mold, thus making the beads, pieces or elements at the edges of the mold and the beads, pieces or elements in the middle of the mold respond more evenly to the steam heat when it is applied.

However, in testing, while there was good consolidation of the pieces, the degree of reanimation (degree to which each piece expands during heating relative to other pieces) varied quite a bit. That is, the VLAP pellets that were allowed to freely rise became relatively huge, but other pellets seemed to barely expand at all. From testing it was discovered, reanimation would be improved if the pellets were agitated within the mold 3.

One example of moving the plurality of expandible elements within the mold during heating and expansion is achieved by blowing air through the steam circuit used to heat the interior of the mold. This creates some movement or agitation within the interior of the mold while the pellets are heated. With more consistent, more complete reanimation, consolidation will also improve (i.e., the desired effect). As is illustrated in FIG. 4, the steam mold 8 allows steam through vents 10 within the mold 8 where the pellets (i.e., the plurality of expandible elements) are situated. To effectuate movement of the VLAP beads, pieces or elements 6 when steam molding, an air jet can (or jet of another gas, e.g., nitrogen) be applied continuously or in a pulsed manner through the same vent 10 that the steam is introduced into the mold, or through a different or separate vent or port in the mold 8.

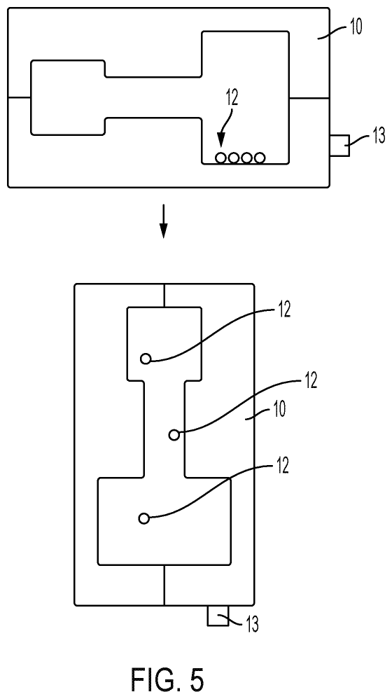

Another example of moving the plurality of expandible elements within the mold is illustrated in FIG. 5 where the entire mold 10 can be rotated during the heating process or at specific times after heating has begun. The movement can be achieved continuously or in periodic increments. Furthermore, the physical movement of the mold 10 illustrated in FIG. 5 need not be rotational. Rather, the physical movement could be achieved using a shaker or some other element that physically moves the mold such that the plurality of expandible elements 12 are moved around in the interior volume of the mold 10. A movement module 13 can be attached to or be attachable to the mold 13 (e.g., a rotiserrie movement, a shaker, etc.) to effectuate movement of the mold 10

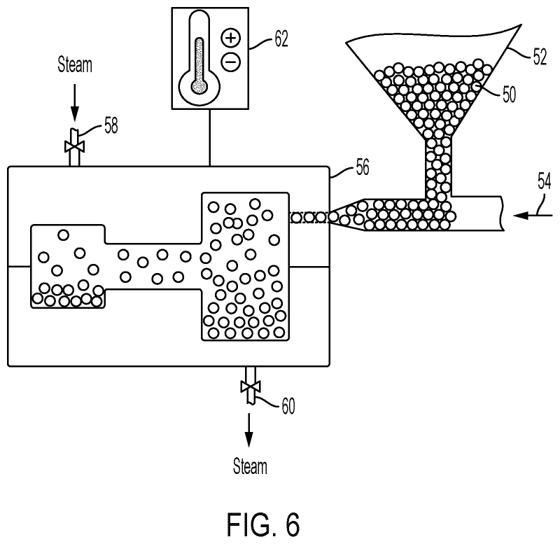

Still another example of moving the plurality of expandible elements with the mold is illustrated in FIG. 6 is to blow the beads (expandible elements 14) into the mold 16, either before or during application of steam heat. This can be accomplished with air jets used alone or in conjunction with steam, as discussed above. Blowing the VLAP beads, pieces or elements into the mold as opposed to blowing them after installation may, in some applications, lead to a more uniform distribution of the VLAP beads, pieces or elements throughout the mold.

A variation this last process is to use a machine like an EPP or EPS injection molding machine. These machines can use steam to activate in their normal operating state to expand each of the plurality of expandible elements, but the expansion is not isotropic (i.e., they generally expand in only one direction). In order to use standard machinery like an EPP or EPS injection molding machine, the expandible elements should have a particle size 5 mm or less (e.g., 2 mm or 1 mm on a side). In exemplary processes using two cycles of steam at 4.25 barg, three dimensional parts have been successfully molded by
1 cycle of pass thru steam (steam injected on one side of the part and then exited on the other side for 15 sec for example, and then the opposite direction for 15 sec for example)
2 1 cycle of steam in autoclave mode where the mold is filled with the steam and the pressure is held for 60 sec for example
3 Demolding the part

Using 1 cycle of steam at 4.25 barg in autoclave mode for 30 sec for example and then demolding

Using 1 cycle of steam at 4.25 barg in pass thru steam mode for 30 sec for example and then demolding

The pressure used generated a minimum temperature on the part of 160° C. as measured with heat-activated temperature strips.

FIG. 6 shows a schematic of the EPS or EPP based process described above, where expandible elements 50 are provided from a storage 52 (shown as a funnel for illustrative purposes only) and are pneumatically conveyed as illustrated by arrow 54 through a port into the mold 56. As explained above, the expansion of the expandible elements 50 is generally not isotropic based on the air pressure causing the expandible elements 50 to be extruded generally individually into a heated the mold 56 through one or more ports. The air pressure 54 also causes the expandible elements 50 to move around within the interior of the mold 56.

In one embodiment, steam may enter the mold 56 through a port with a valved connection 58. The steam will cause expansion of the expandible elements 50 within the mold as well as movement of the expandible elements 50. Steam may exit the mold through another valved port 60. By closing valved port 60 and opening the steam port connection 58, both the temperature and the pressure will increase inside the interior of the mold 56, producing an autoclave like condition which is useful for some applications. In some applications precise temperature control is useful, e.g., depending on the make up of the expandible elements 50, etc. This may be accomplished using a temperature controller 62 which measures the temperature within the mold 56, and permits regulation of one or more of the steam input through connection 58 and steam outlet through connection 60. Steam input can be cycled at various frequencies so as to maintain the temperature throughout the interior of the mold within a preselected temperature range. Control of the steam output can be used to assist in maintaining the temperature within the preselected temperature range (e.g., preventing the temperature from becoming too high or too low for desired processing).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. A method of molding, comprising:
depositing into a mold having an interior volume, a plurality of expandible elements comprised of compressed vertically lapped nonwoven material having binder polymer holding fibers of the vertically lapped nonwoven material in a compressed state, and wherein each of the plurality of expandible elements is smaller than at least one tenth of the interior volume of the mold;
heating the plurality of expandible elements so as to melt the binder polymer and allowing the fibers of the vertically lapped nonwoven material to expand to an expanded state;
moving the plurality of expandible elements to different locations in the interior volume of the mold during heating;
cooling the plurality of expandible elements after heating and moving such that the binder polymer solidifies and holds the fibers in the expanded state, and holds adjacent expandible elements of the plurality of expandible elements together so as to form a molded element; and
retrieving the molded element from the interior volume of the mold,
wherein an amount of the plurality of expandible elements deposited into the mold in the depositing step is sufficient to fill the interior volume of the mold when the molded element is formed during the cooling step.

2. The method of molding as claimed in claim 1 wherein moving is accomplished by moving air through the interior volume of the mold so as to blow the plurality of expandible elements around in the interior volume during molding.

3. The method of molding as claimed in claim 1 wherein moving is accomplished by rotating, shaking, or otherwise moving the mold so as to move the plurality of expandible elements around in the interior volume during molding.

4. The method of claim 1 wherein the depositing, heating, and moving are performed simultaneously by supplying the plurality of expandible elements into the interior of the mold using a stream of heated air.

5. A steam mold, comprising
mold parts which fit together to form an interior volume;
one or more vents or ports which permit steam to be directed into the interior volume; and
a controller for controlling one or more of
continuously and or in pulsed fashion directing air or another gas into the interior volume,
keeping the temperature within the interior volume within a preselected range for a preselected processing time, and
conveying or extruding a plurality of expandible elements into the interior volume of the mold.

6. The steam mold of claim 5 wherein the air or another gas is directed into the interior volume through the one or more vents or permit which permit steam into the interior volume.

7. The steam mold of claim 5 wherein the air or another gas is used to convey or extrude a plurality of expandible elements into the interior volume of the mold.

8. The steam mold of claim 5 wherein the controller controls one or more valves so as to selectively limit steam from exiting the interior of the mold such that the steam directed into the interior volume builds up pressure within the interior volume of the mold.

9. The steam mold of claim 7 wherein in the controller either itself or in combination with a a temperature controller regulates the temperature within the interior volume of the mold within a preselected temperature range.

10. The mold of claim 9 wherein the controller controls a cycle of steam input into the interior of the mold through the one or more vents or ports.

11. The mold of claim 9 wherein the temperature controller controls one or more valves which regulate steam input into the interior volume of the mold and steam output from the interior volume of the mold.
